(19) United States
(12) Patent Application Publication
(10) Pub. No.: US 2006/0106092 A1
(43) Pub. Date: May 18, 2006

(54) Procyanidin B dimers for aromatase inhibition

(76) Inventor: Shiuan Chen, Arcadia, CA (US)

Correspondence Address: PERKINS COIE LLP, POST OFFICE BOX 1208, SEATTLE, WA, 98111-1208, US

(21) Appl. No.: 11/269,129

(22) Filed: Nov. 7, 2005

Related U.S. Application Data
(60) Provisional application No. 60/629,217, filed on Nov. 17, 2004.

(51) Int. Cl. A61K 31/353

(52) U.S. Cl. 514/456

(57) ABSTRACT

Anti-aromatase procyanidin B suppresses estrogen production, thus preventing or treating estrogen-related diseases such as breast tumors, hormone-dependent tumors and hormone-related disorders.

### CROSS-REFERENCE TO RELATED APPLICATION

The present utility application claims priority to U.S. Provisional Application No. 60/629,217 (Chen), filed Nov. 17, 2004, the disclosure of which is incorporated by reference herein in its entirety, including drawings.

### GOVERNMENT INTEREST

The present invention was supported by a NIH-NCCAM pre-doctoral fellowship (F31 AT00059), by NIH grants (ES08258 and CA44735), and the California Breast Cancer Research Program (4PB-0115). The government may have certain rights in the present invention.

### FIELD OF THE INVENTION

The present invention is in the field of use of procyanidin B dimers to prevent and treat breast tumors, hormone-dependent cancers, and hormone imbalances.

### BACKGROUND OF THE INVENTION

Breast cancer (BC) is the most common cancer in American women, affecting approximately 200,000 mothers, sisters, and daughters per year. Nearly 40,000 American women die of BC annually. With a median age at diagnosis of 62, most women are postmenopausal before they are affected with the disease. In breast cancer, in situ estrogen production has been demonstrated to play a major role in promoting tumor growth. Aromatase is the enzyme responsible for the conversion of androgen substrates into estrogens. This enzyme is highly expressed in breast cancer tissue compared to normal breast tissue.

Estrogens play an important role in breast cancer development. Approximately 60% of premenopausal and 75% of postmenopausal patients have estrogen-dependent carcinomas. Aromatase, a cytochrome P450, is the enzyme that synthesizes estrogens by converting C19 androgens to aromatic C18 estrogenic steroids. There is high expression of aromatase in breast cancer tissue and in surrounding stroma when compared to normal breast tissue.¹⁻⁴ When compared to circulating estrogen, in situ produced estrogen has also been shown to play a significant role in breast cancer growth.⁵⁻⁶ Development of a well-tolerated, inexpensive, nutriceutical BC chemopreventive agent for high risk women would have an enormous public health impact, decreasing cancer-associated morbidity, and ultimately saving lives.

### SUMMARY OF THE INVENTION

Procyanidin B dimers are aromatase inhibitors that can be utilized to treat a variety of diseases and disorders associated with increased biological levels of aromatase. Thus, the present invention discloses the anti-aromatase property of procyanidin B dimers and provides a method of preventing or treating diseases or disorders relating to the overexpression of aromatase. The overexpression of aromatase may lead to an increased amount of estrogen in the body of a subject, which is treated by the administration of a procyanidin B. Preferably, the administration of procyanidin B treats estrogen-related diseases and disorders such as breast cancer, hormone-dependent cancers, and hormone imbalances. The method comprises administering a composition comprising procyanidin B, which may be in the form of procyanidin B dimers, to a subject in need thereof in a pharmaceutically acceptable carrier and in a pharmaceutically effective amount. In one embodiment, the administration is repeated to maintain a therapeutically effective amount of the procyanidin B dimer until treatment of the condition is effected. In another embodiment, the subject is a human and the amount of procyanidin B dimers administered to the subject is between 50-500 mg/day and more preferably, between 200-300 mg/day. The procyanidin B dimers may come from any biologically reliable source and may be purified, or be administered as a component of grape seed extract or red wine. Procyanidin B dimers comprise approximately one-third of the mass of grape seed extract. This conversion factor can be used to calculate recommended doses of the procyanidin B dimers.

Another aspect of the invention focuses on suppressing estrogen biosynthesis, which may be desirable for prevention or treatment of any disease or disorder in which estrogen is expressed or over-expressed and is or contributes to the etiology of the disease or disorder. Preferably, procyanidin B dimers are administered to a subject in need of suppressing estrogen biosynthesis. The procyanidin B dimer may have a structure of: 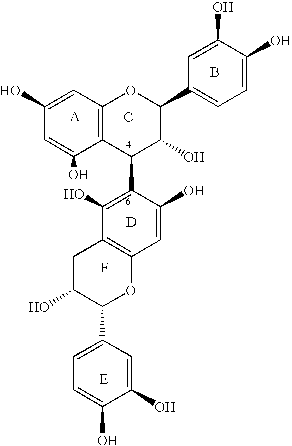
or stereoisomers thereof, or a prodrug thereof, or a pharmaceutically acceptable salt thereof.

The procyanidin B dimers may be administered alone or in conjunction with one or more other therapeutics for treating the relevant disease or disorder. While the procyanidin B dimers can be administered directly to the subject, they may be administered in a pharmaceutically effective carrier. Further, procyanidin B dimers may be administered in a pharmaceutically effective amount. The procyanidin B dimers may be administered to the body at any location in which the therapeutic procyanidin B reaches the intended biological target, which may be administration via an oral, intravenous, parenteral, nasal, or transdermal route.

The procyanidin B compound may be administered in a pure form or as part of a composition. If administered as part of a composition, procyanidin B is may be administered in red wine or grape seed extract. The dosage of grape seed given to a human patient is preferably between 1 mg/day and 1 g/day, more preferably between 20 mg/kg and 750 mg/kg per day, and most preferably between 50 mg/day and 500 mg/day. When the procyanidin B dimers are in grape seed extract, preferably the dosage for a human is between 50-500 mg/day, more preferably, between 50-300 mg/day, and most preferably, between 200-300 mg/day. The grape seed extract used contained approximately 30% procyanidin B dimers. Dosages of grape seed extract containing other concentrations of procyanidin B dimers, such as purer concentrations or pure procyanidin B dimers can be readily calculated.

These and other preferred aspects of the present invention are elucidated further in the detailed description.

### BRIEF DESCRIPTION OF THE DRAWINGS

FIG. 1 shows dose dependent inhibition of human placental aromatase by the chemicals in the 12 HPLC peaks isolated from the polyamide 70% methanol-water fraction. Combo represents the 12 isolated peaks pooled together and evaluated in the aromatase inhibition assay. Data is represented as a percent of the water control.

FIG. 2 is an accurate mass determination of the chemical in HPLC peak 1. The accurate masses for the positive parent ion, M+, and the hydrated positive parent ion, M+H₂O, are shown. These masses were determined based on the masses of sodiated polyethylene glycol standards and potassiated polyethylene glycol standards. The chemical formulas of the standards are indicated in the figure. Exact mass determination of HPLC peak 1 was performed on an electrospray time-of-flight spectrometer Mariner Biospectrometry Workstation with Data Explorer™ software Version 3.2 (PerSeptive Biosystems). The spray tip potential is at 806 and quad and nozzle potential is at 140. Samples were diluted with 90% acetonitrile containing 2% acetic acid. The internal standard used was polyethylene glycol molecular weight 600 (Sigma) which became sodiated or potassiated upon nanospray electrospray ionization (ESI).

FIG. 3 shows the chemical structure of Procyanidin B5. Procyanidins B5-B8 are dimers with the 4-6 linkage and stereoisomers at position C-3. The capital letters in the procyanidin structure represent the standard ring assignments.

FIG. 4 shows the kinetic analysis of HPLC peak 1 procyanidin B dimer in the human placenta microsome aromatase assay. The substrate used was 1β-³H-androstenedione (NEN-Dupont) at 20, 40, 60, 100 and 200 nM. Diamonds are the water control, squares are 5 μM of HPLC peak 1, and triangles are 15 μM of HPLC peak 1. The inset graph is the secondary plot (1/v vs. [I]) used to determine the Kᵢ value for HPLC peak 1 procyanidin dimer.

FIG. 5 shows the effect of the mutations at the active site region of aromatase on the inhibitory activity of HPLC peak 1 procyanidin B dimer. In the presence of various concentrations of HPLC peak 1 procyanidin B dimer, an in vitro microsome assay was performed on the cell homogenates prepared from the wild-type and mutant human aromatase transfected CHO cells. The activities of the untreated samples were taken as 100%. Chinese hamster ovary (CHO) cells transfected with human wild-type and mutant aromatase were grown in Ham's medium, 1 mM sodium pyruvate, 2 mM L-glutamine, 1X-penicillin/streptomycin, 15 mM HEPES, and 10% FBS optimized for CHO cells (Hyclone).

FIG. 6 is a tumor weight comparison of athymic mice post 6 weeks gavage feeding with procyanidin B dimers. Seven to eight week old athymic nude ovariectomized female mice (Charles River Laboratories) were subcutaneously implanted with 5 mg/60 day time-release androstenedione pellets (Innovative Research of America). The feeding started one week after the implantation of androgen pellets. The mice were gavage fed daily for 6 weeks with 100 μl of 1× (Wine 70M-1×) or 3× (Wine 70M-3×) concentrated Pinot Noir polyamide 70% methanol fraction (in water), or water. (The Pinot Noir polyamide 70% methanol fraction contains mainly procyanidin B dimers.) Two weeks after the implantation of androgen pellets, mice were given a 0.2 ml subcutaneously injection in each hind flank containing MCF-7aro cells mixed with equal volume of Matrigel (BD Biosciences) to a final concentration of 1×10⁷ cells/ml. Asterisks indicate significantly different groups compared to water control group (P<0.01). The bars represent the average weight in each group. Statistics were performed using the two-tailed Student's t-test.

FIG. 7 is a graphical comparison showing experimental plots of the weight of tumors in mice for (a) control mice, (b) mice fed a grape seed extract dose of 500 μg, and (c) mice fed a grape seed extract dose of 700 μg.

### DETAILED DESCRIPTION OF THE INVENTION

The present invention has discovered that phytochemicals, which are found in grapes, grapes seeds, red wine and grape seed extract, inhibit aromatase activity in vitro and suppress aromatase-mediated breast tumor formation in vivo. The chemicals in a bioactive fraction isolated from red wine or grape seed extract were identified to be procyanidin B dimers and were shown to be aromatase inhibitors. The most active procyanidin B dimer was found to inhibit aromatase with a Kᵢ value of 6 μM, and the preparation of procyanidin B dimer mixture was found to be much more potent than any one of the individual procyanidin B dimers alone. Using a MCF-7aro tumor induction model, oral intake of the procyanidin B dimer mixture was found to be effective in suppressing aromatase-mediated tumor formation in vivo.

A red wine extract fraction exhibits a potent inhibitory action on aromatase activity. Using UV absorbance analysis, HPLC profiling, accurate mass-mass spectrometry and nanospray tandem mass spectrometry, the compounds in the red wine fraction were identified. Among the compounds identified was procyanidin B dimers, which is found in high levels in grape seeds. Inhibition kinetic analysis on the most potent procyanidin B dimer reveals that it competes with the binding of the androgen substrate with a Kᵢ value of 6 μM. Since mutations at Asp-309, Ser-378 and His-480 of aromatase significantly affected the binding of the procyanidin B dimer, these active site residues are important residues that interact with this phytochemical. The in vivo efficacy of procyanidin B dimers is evaluated in the present invention by an aromatase-transfected MCF-7 breast cancer xenograft model. The procyanidin B dimers reduce androgen-dependent tumor growth, indicating that these chemicals suppress in situ estrogen formation. These in vitro and in vivo studies demonstrate that procyanidin B dimers, such as those in red wine and grape seed extract, can be used as chemopreventive agents against breast cancer by suppressing in situ estrogen biosynthesis.

The intake of the procyanidin B extract by gavage completely abrogated aromatase-induced hyperplasia as well as other changes in the mammary tissue, directly demonstrating the chemopreventive effect of procyanidin B extract against breast cancer by suppressing in situ estrogen formation.

As used in the present invention, “pharmaceutically acceptable salt” may be any non-toxic, acid addition salt formed by a compound of procyanidin B and a pharmaceutically acceptable acid such as phosphoric, sulfuric, hydrochloric, hydrobromic, hydroiodic, citric, maleic, malonic, mandelic, succinic, fumaric, acetic, lactic, metaphosphoric, nitric, sulfonic, p-toluene sulfonic, methane sulfonic acid, and similar. The salts may form hydrates or exist in a substantially anhydrous form. The pharmaceutically acceptable salts have also therapeutic efficacy.

“Stereoisomers” are compound made up of the same atoms bonded by the same bonds but having distinct, non-interchangeable, three-dimensional structures. Stereoisomers of the compounds of the present invention may be in admixture, pure, or substantially pure form. The compounds of the present invention may exist in enantiomeric, diastereomeric, racemic, or other mixtures thereof. “Enantiomers” are stereoisomers whose molecules are nonsuperimposable mirror images of one another and “diastereomers” are stereoisomers that are not mirror images. Separation of racemic mixtures may be achieved using chiral column chromatography, chiral resolving agents, and other well-known separation techniques.

“Prodrugs” as used in the present invention are derivatives of the compounds of the invention which have chemically or metabolically cleavable groups and become the compounds of the invention which are pharmaceutically active in vivo by solvolysis or under physiological conditions.

The term “therapeutically effective amount” as used herein refers to the amount of the anti-aromatase compound that produces a desired therapeutic effect, such as treating the target disease of breast tumors, estrogen-related disorders, hormone imbalances and hormone-dependent tumors. The therapeutically effective amount for each subject will depend upon the activity, pharmacokinetics, pharmacodynamics of the interaction of between the anti-aromatase, such as procyanidin B, and the bioavailable estrogen. It will also depend upon physiological condition of the subject (including age, sex, disease type and stage, general physical condition, responsiveness to a given dosage and type of medication), the nature of pharmaceutically acceptable carrier in a formulation, and a route of administration, among other potential factors. Those skilled in the clinical and pharmacological arts will be able to determine these factors through routine experimentation consisting of monitoring the subject and adjusting the dosage. Remington: The Science and Practice of Pharmacy (Gennaro ed. 20^{th} edition, Williams & Wilkins Pa., USA) (2000).

The term “pharmaceutically acceptable carrier” as used herein means a pharmaceutically-acceptable material, composition or vehicle, such as a liquid or solid filler, diluent, excipient, solvent or encapsulating material, involved in carrying or transporting the anti-aromatase compound in vivo. Each component must be compatible with the other ingredients of the composition and must also be suitable for use in contact with the tissue or organ of humans and animals without excessive toxicity, irritation, allergic response, immunogenecity, or other problems or complications, commensurate with a reasonable benefit/risk ratio.

A “route of administration” for a novel compound or composition can be by any pathway known in the art, including without limitation, oral, enteral, nasal, topical, rectal, vaginal, aerosol, transmucosal, transdermal, ophthalmic, pulmonary, and/or parenteral administration. A parenteral administration refers to an administration route that typically relates to injection. Parenteral administration includes, but is not limited to, intravenous, intramuscular, intraarterial, intraathecal, intracapsular, infraorbital, intracardiac, intradermal, intraperitoneal, transtracheal, subcutaneous, subcuticular, intraarticular, subcapsular, subarachnoid, intraspinal, via intrasternal injection, and/or via infusion.

“Treatment” of or “treating” a disease may mean preventing the disease by causing clinical symptoms not to develop, inhibiting the disease by stopping or reducing the symptoms, the development of the disease, and/or slowing the rate of development of the disease, relieving the disease by causing a complete or partial regression of the disease, reducing the risk of developing the disease, or a combination thereof.

“Therapeutically effective amount” is the amount of novel compound or composition that, when administered to a subject, is effective to bring about a desired effect.

Pharmaceutically acceptable carriers may include sugars, starches, cellulose and its derivatives, malt, gelatin, talc, excipients, glycols, esters, agar, buffering agents, pyrogen-free water, isotonic saline, Ringer's solution, ethyl alcohol, such as red wine, phosphate buffer solutions, and other non-toxic compatible substances employed in pharmaceutical formulations.

Formulations suitable for oral administration may be in any form that can be safely ingested such as capsules, pills, tablets, lozenges, paste, as a solution or a suspension in an aqueous or non-aqueous liquid, syrup, or as pastilles, each containing a predetermined amount of procyanidin B as an active ingredient. In the case of capsules, tablets and pills, the pharmaceutical compositions may also comprise buffering agents. Compressed tablets may be prepared using ingredients well known in the art such as binder, lubricant, inert diluent, preservative, or disintegrant. They may also be formulated so as to provide slow or controlled release of an anti-aromatase compound. Formulations suitable for parenteral administration comprise an anti-aromatase compound in combination with one or more pharmaceutically acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacterostats, solutes which render the formulation isotonic with the blood of the intended recipient or suspending or thickening agents.

The in vitro and in vivo results of the present invention demonstrate that procyanidin B dimers in red wine can be used as chemopreventive agents against breast cancer, other estrogen-related cancers, and hormone imbalances by suppressing in situ estrogen biosynthesis. Five ml of red wine can produce 100 μl of 1× fraction. A typical conversion factor for determining mouse to human dosage of chemotherapeutic agents is 25, calculated based on body surface area.¹¹ Well-tolerated daily doses of grape seed extract (GSE) comprising procyanidin B are 50-500 mg/day, and that the degree of estrogen suppression is related to dose of GSE. The present invention has discovered that consumption of 125 ml of red wine per day would provide adequate amounts of procyanidin B dimers to suppress in situ aromatase in an average postmenopausal woman. However, a subject with a disease or disorder that has progressed may require higher doses of procyanidin B.

Thus, there is a dose effect between both procyanidin B dimers and suppression of estrogen biosynthesis and procyanidin B dimers significantly suppressing serum estrogens in healthy postmenopausal women at increased risk for developing breast cancer. Among the benefits of procyanidin B dimer as an aromatase inhibitor are the characteristics that it is potent and highly effective at reducing serum estrogen levels, selective for the aromatase enzyme, easily attainable, easy to administer, and non-toxic. In fact, no adverse effects were reported in any of these studies. Mouse and rat studies have used doses up to one gm/kg/day for 90 days with no observable adverse effects noted.⁷

Isolation of anti-aromatase chemicals from red wine. In order to better understand the molecular nature of red wine's anti-aromatase activity, the chemicals that are capable of inhibiting aromatase were isolated and characterized. Whole red wine was applied to the polyamide column (Discovery DPA-6S SPE Supelco, Bellefonte, Pa.). The elution solvent consisted of a step-gradient of methanol and water. This particular SPE column produced fractions exhibiting a range of inhibitory activity with the best inhibitory fractions seen with 60-80% methanol-water gradients.

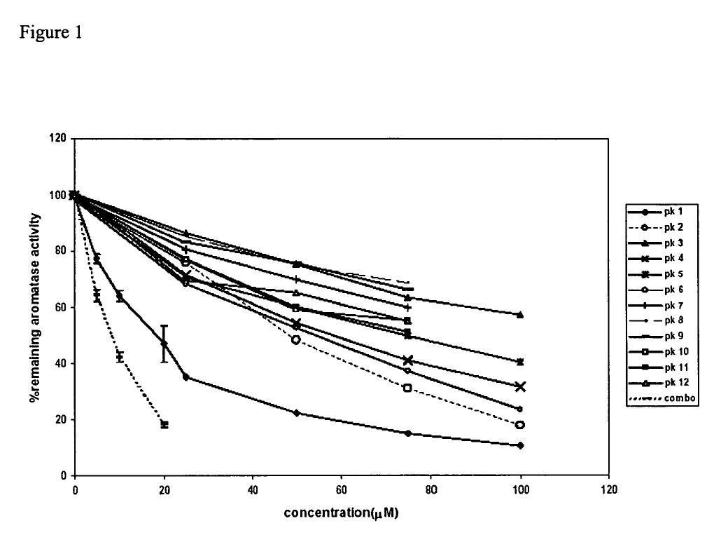

When the active polyamide fractions were injected onto a reverse phase high performance liquid chromatography (HPLC) system, 12 peaks were obtained. The UV maximal absorbances of HPLC peaks 1-6 and 8 were between 279-280 nm. Both HPLC peaks 9 and 12 had UV absₘₐₓ of 264 nm. Compounds found in HPLC peaks 1-6 and 8 had similar UV absorbance characteristics to procyanidins B1 and B2 that had UV absₘₐₓ of 279 to 279.5 nm and to catechin and epicatechin standards which had UV absₘₐₓ of 277.5 to 278.5 nm. Peaks 9 and 12 were similar to gallic acid that also had an UV absₘₐₓ of 264 nm. Once concentrations were determined for the twelve unknown compounds described below, they were evaluated for their ability to inhibit aromatase in human placental microsomes. Increasing concentrations of each HPLC peak compound was assayed in the presence of 100 nM androstenedione substrate. The aromatase activity results show that all HPLC fractionated compounds inhibited aromatase in a dose-dependent manner (FIG. 1). The most effective inhibition curve was observed with HPLC peak 1 whose IC₅₀ was between 15-20 μM (calculated using a molecular weight of 579). The reconstituted mixture was significantly more effective than any single chemical as clearly shown in FIG. 1.

Accurate Mass-Mass Spectrometry. Since HPLC peak 1 chemical had the best inhibitory activity on aromatase, a Mariner time-of-flight mass spectrometer was used to determine the precise mass or m/z for better estimating the elemental composition. To calibrate every run, an internal standard was added to each sample. Since the UV spectral analysis suggested that the compound might be a procyanidin, the internal standard used was polyethylene glycol, mw 600 (PEG600) which has a well-established mass spectra profile and contains product ions that borders the m/z=579 (the molecular weight of a procyanidin dimer) without overlapping at the same position. The Mariner Data Explorer™ software performs a mass calibration based on the internal standard. Once an exact mass had been determined, the Data Explorer™ software also calculated the possible elemental compositions and scored them based upon statistical probability. The unknown compound HPLC peak 1 combined with PEG600 was analyzed as the internal standard in the Mariner mass spectrometer.

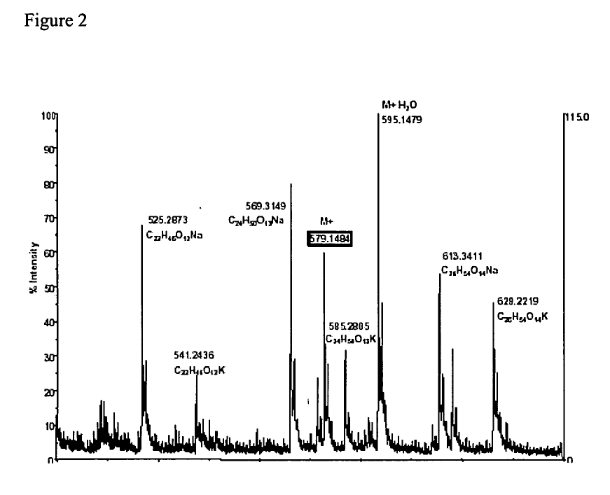

The observed positive parent ion mass (M+) for HPLC peak 1 was 579.1484 that represented an accurate mass with an error of just 3 ppm when compared to the theoretical mass for a procyanidin B dimer (FIG. 2). For HPLC peak 1 chemical, the elemental analysis gave the most likely chemical formula as C₃₀H₂₇O₁₂ with an 89.1% isotope match score. In practice, a compound's mass measured with an error of less than 10 ppm from the theoretical mass is considered to be an exact mass. Therefore, HPLC peak 1 exhibited a mass (M+) of 579 and an elemental formula of C₃₀H₂₇O₁₂ which confirmed that HPLC peak 1 is a procyanidin B dimer.

The procyanidin B dimers consist of 8 stereoisomers composed of catechin and epicatechin monomers that can be divided into 2 subgroups based upon their 4-8 or 4-6 linkages. Only three procyanidin B dimers are commercially available: procyanidin B1, B2 and B3. Using analytical RP-HPLC, procyanidins B1, B2 and B3 were found to coelute with HPLC peaks 2, 6 and 3, respectively. Therefore, peak 1 is a procyanidin B dimer that is different from procyanidin B1-B3.

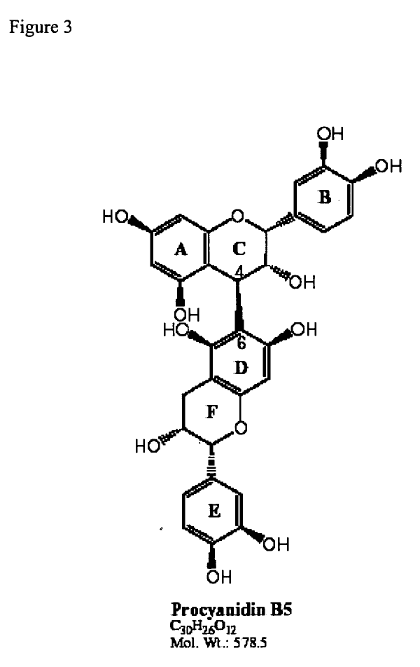

The HPLC fractions were also analyzed by electrospray ionization mass spectrometry (ESI-MS) with collision-induced dissociation (CID). Tandem fragmentation and ionization of the parent ion can help elucidate chemical and structural features of the molecule. As expected, HPLC peaks 2, 3 and 6 had identical tandem MS profiles up to MS³ as did procyanidin B1, B3 and B2. HPLC peaks 1 and 5 had a different fragmentation pattern from HPLC peaks 2, 3 and 6 as seen by their MS² fragment ion of 301 compared to MS² fragment ion of 427. There is no noticeable difference between dimers B1, B2 or B3 because they all belong to the same subgroup of procyanidin dimers with the 4-8 linkage. Thus, HPLC peaks 1 and 5 may be procyanidin B dimers with the 4-6 linkage. (No compounds from the 4-6 linkage subgroup are commercially available.) Mass spectral analyses has determined that the major chemicals in the 60-80% methanol eluted polyamide fraction are isomers of procyanidin B dimer. HPLC peak 1 appeared to be very likely a procyanidin B dimer with 4-6 linkage (See FIG. 3). NMR analysis was carried out to confirm that peak 1 is, in fact, a procyanidin B dimer.

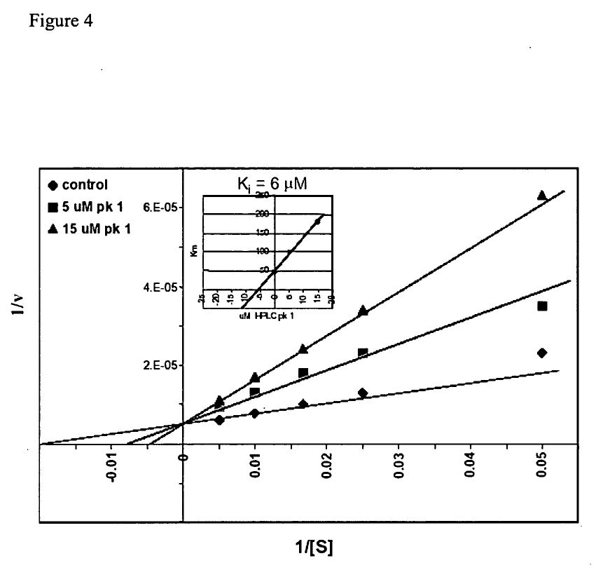

Enzyme Kinetic Analysis for HPLC Peak 1 Procyanidin Dimer. To elucidate the molecular basis of peak 1 procyanidin B dimer inhibition of aromatase, enzyme kinetic analysis was performed using the human placenta microsome aromatase assay. The results show that the dimer from HPLC peak 1 inhibited aromatase in a competitive manner with respect to the substrate (FIG. 4). A Kᵢ value of 6 μM for HPLC peak 1 was determined from the secondary plot (1/v versus [I]). Therefore, this procyanidin B dimer inhibits aromatase/estrogen biosynthesis in a specific manner with a Kᵢ value similar to the known aromatase inhibitor, aminoglutethimide.⁸

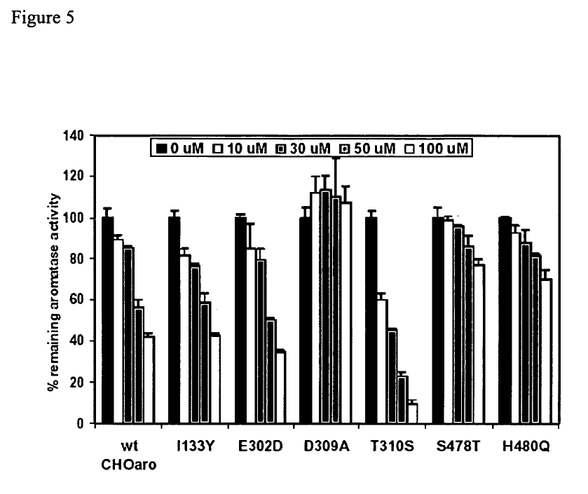

Aromatase Site-Directed Mutagenesis Studies. The specificity of the interaction of peak 1 procyanidin B dimer with aromatase was further evaluated using aromatase active site mutants. The wild-type and six human aromatase mutants (I133Y, E302D, D309A, T310S, S478T and H480Q) were used. Previous studies performed by the present inventors have determined that these mutated amino acids are present in the active site of aromatase.⁹ With increasing concentrations of HPLC peak 1 procyanidin B dimer, the present analysis revealed that mutations I133Y and E302D did not alter the inhibition profile obtained from HPLC peak 1 when compared to the wild-type aromatase (FIG. 5).

Mutant D309A produced the most dramatic effect by completely eliminating the ability of HPLC peak 1 dimer to inhibit aromatase. Two other mutants S478T and H480Q also proved to be significant in reversing the inhibitory action of HPLC peak 1. Interestingly, the T310S mutant produced a more effective dose response upon treatment with HPLC peak 1 procyanidin B dimer when compared to the wild-type aromatase. These results support the finding from enzyme kinetic analysis that this compound binds to the active site of aromatase and indicate that Asp-309, Thr-310, Ser-478 and His-480 are involved in the interaction with the peak 1 procyanidin B dimer.

In vivo Experiments. In vitro studies of the present invention have found that most of the chemicals in the 60-80% methanol eluted polyamide fraction are procyanidin B dimers, which are inhibitors of aromatase. It was also discovered that the mixture was more active than individual chemicals. Animal experiments using the entire polyamide 60-80% methanol-water fraction that contains mainly procyanidin B dimers were performed, which may also be performed with purified procyanidin B dimers.

The aromatase inhibitory action of the polyamide Pinot noir wine extract, which comprises procyanidin B dimers, was first analyzed in a mouse xenograft model using BALB/c Nu-Nu athymic intact (with ovaries) mice. Mice were given two subcutaneous injections (0.2 ml per site) of MCF-7aro cells diluted in equal volume of Matrigel to a final concentration of 1×10⁷ cells/ml. MCF-7aro cells are ER positive and over-express aromatase.¹⁰ These cells proliferate in an androgen-dependent manner.

It was found that mice fed daily with the polyamide 70% methanol-water fraction (70M) at 50 μl and 100 μl doses had a significant reduction in tumor growth when compared to the control (P<0.03). The average tumor weights for each group were as follows: 58.5±23.2 mg (control), 44.8±30.5 mg (70M wine extract 25 μl), 36.4±25.9 mg (70M wine extract 50 μl), and 33.3±26.8 mg (70M wine extract 100 μl). The ovaries and uteri were also removed in order to examine any effects from the wine extract on other endocrine glands that either produce estrogen (ovaries) or require estrogen for its cell maintenance (uterus). The average ovary-uterine weights for each group were as follows: 172.8±16.2 mg (control), 170.1±31 mg (70M wine extract 25 μl), 131.4±44 mg (70M wine extract 50 μl), and 134.1±41 mg (70M wine extract 100 μl). Again, the ovary-uterine weights of mice fed daily with the polyamide 70% methanol-water fraction at 50 μl and 100 μl were significantly smaller than those of the control mice.

Serum from each mouse was also collected and analyzed for estradiol and estrone concentrations. Mice treated with increasing concentrations of wine extract showed a decreasing trend in the levels of estradiol (E2) and estrone (E1) compared to the control mice. The average estrogen concentration values for wine extract treated mice showed a clear, but statistically ambiguous, difference when compared to mice treated with water. The large error bars may be due to the intact ovaries in the mice that contribute to the variation in circulating estrogen levels. Over the course of the two-month experiment, the weights of animals in each group were recorded where all groups had similar body weight.

In order to eliminate the effect of estrogens generated endogenously by the ovary, in the second type of animal experiments, athymic nude ovariectomized female mice were used. Additionally, a more concentrated polyamide wine extract was used in these experiments, at 100 μl each of a 1× or 3× concentrated extract was used as compared to just an 1× concentrate used at different volumes (25, 50, 100 μl). All mice received a 5 mg/60-day release androstenedione pellet that was implanted subcutaneously. For 6 weeks, mice were gavage fed daily with either 100 μl of 1× or 3× concentrated 70M wine extract, or water. After the first week of gavage treatment, each mouse was given a 0.2 ml subcutaneous injection into each hind flank containing MCF-7aro cells suspended in equal volume Matrigel to a final concentration of 1×10⁷ cells/ml.

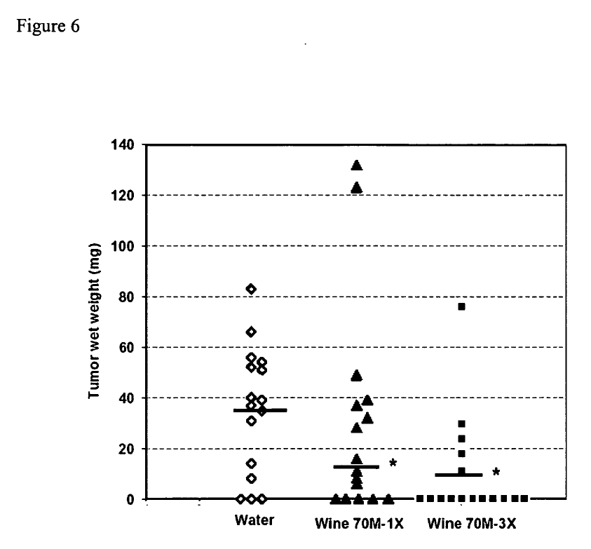

At the completion of 6 weeks of gavage treatment, mice were sacrificed and blood, tumor and uteri were removed, weighed and sent out for histological evaluation. Mice treated with 1× and 3× concentrate 70M wine extract showed a significant reduction (P<0.01) in tumor size compared to the androstenedione control mice fed with water (15.7±16.2 mg and 9.9±20.2 mg vs. 35.4±25.2 mg, respectively) (FIG. 6). A more detailed analysis of individual mice per treatment group is summarized in Table 1.

The positive control group, And+/H₂O, was the group that had the highest percentage (62.5%) of mice having tumor growth on each flank. Fifty percent of the 1× concentrate treated group had growth of two tumors. While the group with the lowest number of mice with two tumors was the mice treated with the 3× concentrate. The 3× concentrate treated mice had the most dramatic suppression of tumor growth as indicated by the high percentage (62.5%) of animals which had no MCF-7aro growth at either injection site. Interestingly, only 12.5% of the 1× treated mice had complete tumor suppression while 37.5% of the mice had just one tumor with an average tumor weight of 22.7 mg.

TABLE 1
Summary of tumor numbers in individual athymic nude mice gavage
fed with Pinot Noir polyamide 70% methanol fraction.

| | | | | Percentage of |
|---|---|---|---|---|
| | | Tumors on | No tumors | injected |
| Treatment | Tumors on | 1 side | on both | sites with |
| Group | 2 sides | only | sides | tumors |
| And+^{a}/H₂O | 5^{b} (62.5%^{c}) | 3 (37.5%) | 0 | 81.3% |
| (n = 8) | | | | |
| And+/Wine 1× | 4 (50%) | 3 (37.5%) | 1 (12.5%) | 68.8% |
| (n = 8) | | | | |
| And+/Wine 3× | 2 (25%) | 1 (12.5%) | 5 (62.5%) | 31.3% |
| (n = 8) | | | | |

^{a}= androstenedione implanted pellet
^{b}= number of animals per group
^{c}= percentage of mice in group

Further testing of powdered grape seed extract (GSE) (Activin IH636, Dry Creek Nutrition) in a nude mouse model revealed that the weights of tumors from mice fed 750 ug/day for 6 weeks were 52% of those from the control mice (p=0.0004).

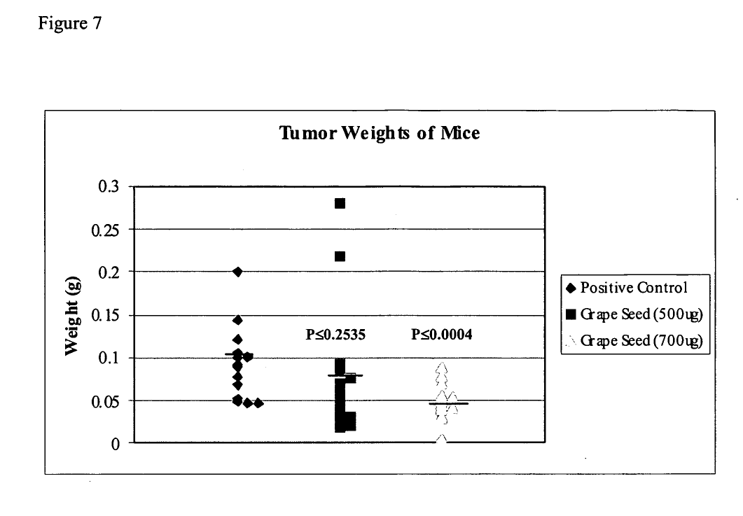

To evaluate whether these treatments caused any deleterious effects upon other endocrine glands, the uteri were removed and weighed. The wet uterine weights of all wine extract treated mice were not found to be statistically different from the uteri of androstenedione control mice. Blood samples were taken and serum analyzed for the circulating estrogen. The concentrations of circulating estrogen in ovariectomized mice were found to be significantly lower than that found in intact animals (with ovary). Furthermore, the results suggested only a slight trend in reduced blood estrogen concentrations upon treatment with increasing concentrations of wine extract. The results from uterine weight measurements and circulating estrogen levels support the hypothesis that in situ produced estrogen plays a larger role in aromatase expressing and estrogen-receptor positive breast tumor growth than circulating estrogen. The results of another experiment examining mice tumor weights at various concentrations of grape seed extract, shown in FIG. 7, further support this finding.

Since procyanidin B dimers are competitive inhibitors, not irreversible inhibitors, the specific aromatase activity (per tumor weight) should be the same when measured on tumors which have been removed from mice. There was not any difference detected in specific enzyme activity by measuring aromatase activity in homogenized tumor tissue from non-treated or treated mice.

These mice tumors were further evaluated using immunohistochemistry with M30 CytoDEATH mouse monoclonal antibodies that bind to an early apoptotic marker, the caspase cleaved product of human cytokeratin 18 (CK18) cytoskeletal protein. The M30 CytoDEATH antibody recognizes a specific caspase cleavage site in cytokeratin 18 that is not detected in native CK18 of normal cells. The tumors of mice treated with wine extract showed a slight increase in the number of apoptotic cells when compared to tumors of control mice, however, the difference did not appear to be statistically significant. These results demonstrate that the observed reduction in tumor growth in the wine extract treated mice was due to inhibition of aromatase and not due to a non-specific cytotoxic effect.

The effect of polyamide wine extract in a MCF-7 breast cancer xenograft model has also been examined. In these in vivo studies, athymic nude ovariectomized female mice were given two subcutaneous injections of MCF-7 instead of MCF-7aro cells, and the tumor formation was induced using 5 mg/60-day release 17β-estradiol pellet implanted subcutaneously. Other experimental conditions were identical to those described for the experiments using MCF-7aro cells. MCF-7 cells have minimal aromatase activity and this is the reason that anti-aromatase compounds did not affect the tumor formation. It was found that 3× concentrated 70M wine extract fed daily at 100 μl was not able to suppress the growth of MCF-7 tumors (p=0.286). The average tumor weights for the estrogen control and 3× concentrated extract treated mice were 44.2±60.3 mg vs. 52.4±45.9 mg, respectively. The weights of the tumors from mice without estrogen pellet were 21.0±15.4 mg. These results are crucial for confirming the anti-aromatase activity of procyanidin B dimers, which may be found, among other places, in red wine, grape seed extract, or in purified form. Procyanidin B dimers have a highly specific inhibitory activity against aromatase/estrogen biosynthesis.

To further test the efficacy of the methods, serum sex steroid hormone assays may be performed in humans. Total Estradiol (E₂) is determined by incubating serum with ³H-E₂ and extracted with hexane and ethyl acetate to remove unconjugated steroids. After evaporating under nitrogen, the residue is redissolved in isooctane and applied on a column of Celite impregnated with ethylene glycol. E₂ is eluted in ethyl acetate in isooctane and redissolved in buffer. Then it is incubated with ¹²⁵I-E₂ and anti-E₂ serum. Antibody-bound and unbound ¹²⁵I-E₂ are separated by adding a second goat anti-rabbit antibody, centrifuging, and aspirating the supernatant. Total estradiol is then quantitated using a gamma counter. Bioavailable E₂ is calculated by determining the sex hormone binding globulin (SHBG) level and subtracting that from the total estradiol level. SHBG is measured by a solid-phase, two-site chemiluminescent immunoassay using the Immulite analyzer.

Alkaline phosphatase conjugated anti-SHBG polyclonal antibodies are introduced into the reaction tube, and the tube is incubated for 30 minutes at 37° C. SHBG in the sample is bound, forming an antibody sandwich complex. Unbound conjugate is then removed by a centrifuge wash, after which the chemiluminescent substrate is added, and the reaction tube is incubated for another 5 min. The chemiluminescent substrate undergoes hydrolysis in the presence of alkaline phosphatase, yielding an unstable intermediate. Continuous production of the intermediate results in the sustained emission of light. The light is measured by an illuminometer and is proportional to the concentration of SHBG in the sample. Estrone (E1) is determined similarly to E₂ but with incubation with ³H-E1. Estrone Sulfate (E₁S) is determined with incubation with ³H-E₁S and extraction with hexane and ethyl acetate, followed by deproteinization with methanol. After evaporating under nitrogen, the residue is reconstituted with sodium acetate buffer and hydrolyzed with arylsulfatase. The hexane:ethyl acetate extraction step is repeated, the extract is redissolved in buffer, and then the E₁ RIA is carried out.

The assay sensitivities are: E₂=5 pg/ml=1.36 pmol/L, E₁=5 pg/ml=1.35 pmol/L, and E₁S=0.16 ng/ml. Previous studies of aromatase inhibitor testing in normal postmenopausal females reported mean baseline E₂ levels of 21-46 pmol/l, with mean post-treatment suppression levels in the 2.7-3.3 pmol/l range. They also reported mean baseline E₁ S levels of 476-561 pmol/l, which dropped to 32-40 pmol/l after treatment.¹²⁻¹³ Therefore, the level of estrogen suppression with an aromatase inhibitor is well within the detectable range using these assays. Intra-assay and inter-assay coefficients of variation (CV) for all four assays are well below 15%.

Another measure of the efficiency is examination of bone marker metabolism. Biochemical markers of bone metabolism such as these can mark changes over as little as 1-3 months. Serum bone-specific alkaline phosphatase (BSAP), a marker of bone formation, is measured using a solid phase two-site immunoradiometric assay (IRMA). The sensitivity of the assay is 2.0 μg/L. The expected postmenopausal range is 14.2-42.7 μg/L. Urine N-telopeptide crosslinks (NTX), a marker of bone resorption, is measured using a competitive-inhibition enzyme-linked immunosorbent assay (ELISA). Final values are reported as bone collagen equivalents (BCE) in nM/L corrected for creatinine clearance in mM/L. The expected range in postmenopausal women is 26-124 nM BCE/mM creatinine.

Another test performed is determining the cortisol level after ACTH stimulation, which is done as a marker of nonspecific adrenal suppression. Nonspecific adrenal suppression is an indication of aromatase inhibitors. An indwelling antecubital venous cannula is placed, and a pretest blood sample is taken. Another sample is taken 30 minutes after administering 25 units Cosyntropin I.V. An afternoon baseline cortisol level should be <20 μg/dL. The post Cosyntropin level should be >20 μg/dL and at least 7 μg/dL higher than the baseline level. A lack of an adequate stimulatory response suggests primary adrenal insufficiency that may occur as a consequence of nonspecific adrenal suppression from aromatase inhibitors.

In parallel, aliquots of each plasma sample are also extracted and tested for its ability to inhibit aromatase in vitro. A placental microsome aromatase assay can be used to determine the aromatase activity in patient plasma. ³H₂O-labeled androst-4-ene-3,17-dione is added to a mixture of aromatase-rich human placental microsomes, progesterone, NADPH, and a tissue-nutritive solution. The level of aromatase activity is determined by measuring the amount of ³H2O released from [1b-3H]-androstenedione substrate after timed incubation with GSE. The amount of ³H2O present is quantitated in the aqueous phase following extraction of the androgen substrate with dextran-coated charcoal. The lower this value, the stronger the aromatase inhibition. The parallel analysis of procyanidin dimer levels and aromatase inhibition allows comparison between the pharmacokinetic data and the pharmacodynamic data to determine whether the time course or extent of enzyme inhibition is correlated with the circulating level of any one of the individual procyanidin dimers.

### Experimental Methods

Preparation of wine fractions. One hundred ml of complete red wine (Pinot Noir, Hacienda, Sonoma, Calif. 1999) was applied to each 5 g/60 ml capacity polyamide column (Discovery DPA-6S SPE Supelco). Fractions were eluted by a step-gradient (50 ml each step) of increasing methanol to water. The 70% methanol-water fraction (50 ml) was rotor-evaporated to dryness and then redissolved in 2 ml deionized water to produce the 1× extract or 0.67 ml water for the 3× concentrate.

The HPLC system used was a Beckman Gold System Programmable Solvent Module 126 with a Shimadzsu SPD-6A UV spectrophotometric detector set at 214 nm. The active wine extract was separated on a Discovery C18 column (5 μM, 25 cm×4.6 mm) (Supelco) using a shallow gradient at a 1 ml/min flow rate with solvent A (0.1% TFA, Pierce) and solvent B [90% acetonitrile (Burdick and Jackson), 0.1% TFA]: 5-10% B (5 min), 10-30% B (60 min) and 30-98% B (15 min).

Mass Spectrometry. The accurate mass-mass spectral analysis was performed using a Mariner time-of-flight mass spectrometer (Perseptive Biosystems, Framingham, Mass.). Tandem mass spectrometry of isolated HPLC peaks was performed on a LCQ quadropole ion trap mass spectrometer (Thermo-Finnigan, San Jose, Calif.) through electrospray ionization mass spectrometry (ESI-MS).

In vitro aromatase Assay. Aromatase activity was determined using the tritiated water release method.⁸ The assay was performed using placental microsomes or microsomal fractions prepared from aromatase-expressing Chinese hamster ovary (CHO) cells. The assay reaction mixture (500 μl) contained the substrate [1β-³H(N)] androst-4-ene-3,17-dione (specific activity 24.7 Ci/mmol) (100 nM), microsomal preparations (20 μg), progesterone (10 μM), bovine serum albumin (0.1%) and potassium phosphate (67 mM, pH 7.4). Progesterone was required to suppress endogenous 5α-reductase in the cell homogenates that also consume the androgen substrate. After incubation for 10 minutes at room temperature, 50 μl of NADPH (12 mM) was added to the mixture and incubated in a 37° C. water bath for 10 minutes. At the end of 10 minutes, the reaction was stopped by the addition of 500 μl of 5% TCA. After a 10 minute centrifugation at 1000×g, supernatants were removed to new glass tubes and mixed with an equal volume of chloroform to remove unreacted substrate.

For the second extraction, the upper aqueous phase was transferred to microfuge tubes containing a dextran-charcoal pellet. Charcoal mixtures were vortexed and subsequently pelleted by centrifugation at 15,000×g for 5 minutes. For each sample, a 300 μl aliquot of the supernatant containing the tritiated water product, was mixed with 3 ml Scintisafe 30% liquid scintillation cocktail and counted in a Tri-Carb Liquid Scintillation Analyzer 1600CA (Packard, Downers Grove, Ill.).

To determine the aromatase suppression activity of wine chemicals, the assay was performed in the presence of wine fractions at the indicated amounts. Inhibition kinetic analysis on wine chemicals was performed with varying concentrations of the substrate androstenedione at 20, 40, 60, 100 and 200 nM.

In vivo Experiments. In the experiments using intact animals, 5-6 week old female BALB/c Nu-Nu, athymic, nonovariectomized mice were purchased (Charles River Laboratories). At about 8 weeks of age, mice were subcutaneously implanted with a 5 mg/60 day time-release androstenedione pellets (Innovative Research of America, Sarosota, Fla.). A week later, mice were individually gavage fed with 100 μl water control, or 25, 50 or 100 μl of a 1× concentrated wine polyamide extract (in water). Each animal received daily gavage treatment for 42 consecutive days. At 10 weeks old, mice were given two subcutaneously injections of MCF-7aro cells. These cells were grown in Eagle's MEM with non-essential amino acids, sodium pyruvate, and Earle's salts in 10% fetal calf serum. The MCF-7aro cells were harvested and resuspended in an equal volume of Matrigel (BD Biosciences) to a final concentration of 1×10⁷ cells/ml. Body weights were monitored weekly as an indicator of the animals overall health. At the end of 6 weeks of gavage treatment, mice were euthanized, blood samples were collected, and tumors and ovaries-uteri were removed, weighed, and sent for hematoxilin and eosin (H & E) histological staining.

Estrogen concentrations were determined from mouse sera. Mouse blood was obtained through cardiac puncture and immediately combined with heparin. Whole blood was separated to serum and plasma in a tabletop centrifuge. Sera was frozen until later evaluation by radio-immunoassay for estrogen and estrone levels.

For the animal experiments using ovariectomized animals, athymic nude ovariectomized female mice were used (Charles River Laboratories). Mice also received the same subcutaneous androstenedione 60 day time-release pellet implants as in the previous experiment. Control animals were given androstenedione pellet and gavage fed 100 μl of sterile water. Treatment groups were given androstenedione pellets and either 1× or 3× concentrate of the Pinot Noir polyamide 70% methanol fraction in a 100 μl volume. All other parameters i.e. number of cells injected, duration of experiment, and experimental analyses were identical to the previously described animal experiment using intact mice. For the animal experiments using MCF-7 cells (the cells without aromatase), MCF-7 cells and estrogen pellets, instead of androgen pellets, were used. Other conditions were identical to those described for the experiments using MCF-7aro cells.

All references cited herein are hereby incorporated in their entirety by reference. The foregoing is merely illustrative of the principles of this invention and various modifications can be made by those skilled in the art without departing from the scope and spirit of the invention.

### CITED REFERENCES

[1] Esteban, J., Warsi, Z., Haniu, M., Hall, P., Shively, J. E. & Chen, S. Detection of intratumoral aromatase in breast carcinomas. An immunohistochemical study with clinicopathologic correlation. Am. J. Pathol. 140, 337-343 (1992).
[2] Bulun, S. E., Price, T. M., Mahendroo, M. S., Aitken, J. & Simpson, E. R. A link between breast cancer and local estrogen biosynthesis suggested by quantification of breast adipose tissue aromatase cytochrome P450 transcripts using competitive polymerase chain reaction after reverse transcription. J. Clin. Endocrinol. Metab. 77, 1622-1628 (1993).
[3] Santen, R. J., Martel, H., Hoagland, M., Naftolin, F., Roa, L., Harada, N., Hafer, L., Zaino, R. & Santner S. J. Stromal spindle cells contain aromatase in human breast tumors. J. Clin. Endocrinol. Metab. 79, 627-632 (1994).
[4] Lu, Q., Nakmura, J., Savinov, A., Yue, W., Weisz, J., Dabbs, D. J., Wolz, G. & Brodie, A. Expression of aromatase protein and messenger ribonucleic acid in tumor epithelial cells and evidence of functional significance of locally produced estrogen in human breast cancers. Endocrinology 137, 3061-3068 (1996).
[5] Tekmal, R. R., Ramachandra, N., Gubba, S., Durgam, V. R., Mantione, J., Toda, K., Shizuta, Y. & Dillehay, D. L. Overexpression on int-5/aromatase in mammary glands of transgenic mice results in the induction of hyperplasia and nuclear abnormalities. Cancer Res. 56, 3180-3185 (1996).
[6] Yue, W., Wang, J. P., Hamilton, C. J., Demers, L. M. & Santen, R. J. In situ aromatization enhances breast tumor estradiol levels and cellular proliferation. Cancer Res. 58(5), 927-932 (1998).
[7] Ray, S., et al., Acute and long-term safety evaluation of a novel IH636 grape seed proanthocyanidin extract. Res Commun Mol Pathol Pharmacol, 2001. 109(3-4): p. 165-97.]
[8] Thompson, E. A. Jr. & Siiteri, P. K. Utilization of oxygen and reduced nicotinamide adenine dinucleotide phosphate by human placental microsomes during aromatization of androstenedione. J. Biol. Chem. 249, 5364-5372 (1974).
[9] Kao, Y.-C., Cam, L. L., Laughton, C. A., Zhou, D. & Chen, S. Binding characteristics of seven aromatase inhibitors to human aromatase. A site-directed mutagenesis study. Cancer Res. 56, 3451-3460 (1996).
[10] Zhou, D., Pompon, D. & Chen, S. Stable expression of human aromatase cDNA in mammalian cells-a useful system for aromatase inhibitor screening. Cancer Res. 50, 6949-6954 (1990).
[11] Devita, V. T., Rosenberg, S. A. & Hellman, S. Cancer: Principles and Practice of Oncology, 2^{nd} Edition (Lippincott Williams & Wilkins), 273-274 (1993).
[12] Yates, R. A., et al., Arimidex (ZD1033): A Selective Potent Inhibitor of Aromatase in Postmenopausal Female Volunteers. Br J Cancer 1996; 73(4): 543-548.
[13] Dowsett, M., et la., Effects of Aromatase Inhibitor Anastrozole on Serum Oestrogens in Japanese and Caucasian Women. Cancer Chemother Pharmacol, 2000, 46(1): 35-39.

### OTHER REFERENCES

Johnston, S. R. D., Smith, I. E., Dowsett, M. Place of aromatase inhibitors in the endocrine therapy of breast cancer. In Aromatase Inhibition and Breast Cancer. (eds. Miller, W. R. & Santen, R. J.) (Marcel Dekker Inc., New York, 2001).
Winer, E. P., Hudis, C., Burstein, H. J., Chlebowski, R. T. et al. American Society of Clinical Oncology technology assessment of the use of aromatase inhibitors as adjuvant therapy for women with hormone receptor-positive breast cancer: Status report 2002. J. Clin. Oncol. 20, 3317-3327 (2002).
Ellis, M. J., Coop, A., Singh, B., Mauriac, L. et al. Letrozole is more effective neoadjuvant endocrine therapy than tamoxifen for ErbB-1 and ErB-2-positive, estrogen receptor-positive primary breast cancer: Evidence from a phase III randomized trial. J. Clin. Oncol. 19, 3808-3816 (2001).
Smith-Warner, S. A., Elmer, P. J., Tharp, T. M., Fosdick, L., Randall, B., Gross, M., Wood, J. & Potter, J. D. Increasing vegetable and fruit intake: Randomized intervention and monitoring in an at-risk population. Cancer Epidemiol. Biomark. Prev. 9, 307-317 (2000).
Trichopoulou, A., Lagiou, P., Kuper, H. & Trichopoulos, D. Cancer and Mediterranean Dietary Traditions. Cancer Epidem. Biomark. Prev. 9, 869-873 (2000).
Dos Santos, S. I., Mangtani, P., McCormack, V., Bhakta, D., Sevak, L. & McMichael, A. J. Lifelong vegetarianism and risk of breast cancer: a population-based case-control study among South Asian migrant women living in England. Int J Cancer 99 (2), 238-244 (2002).
Chen, S., Sun, X.-Z., Kao, Y.-C., Kwon, A., Zhou, D. & Eng, E. Suppression of breast cancer cell growth with grape juice. Pharmaceutical Biology 36, Suppl, 53-61 (1998).
Eng, E. T., Williams, D., Mandava, U., Kirma, N., Tekmal, R. R. & Chen, S. Suppression of aromatase (estrogen synthetase) by red wine phytochemicals. Breast Cancer Res Treat 67, 133-146 (2001).
Renaud, S. & de Lorgeril, M. Wine, alcohol, platelets, and the French paradox for coronary heart disease. Lancet 53, 22-26 (1992).
Criqui M. G. & Ringel B. L. Does diet or alcohol explain the French paradox? Lancet 344, 1719-1723 (1994).
Vachon, C. L., Kushi, L. H., Cerhan, J. R., Kuni, C. & Sellers T. A. Association of diet and mammographic breast density in the Minnesota breast cancer family cohort. Cancer Epidemiol. Biomark. Prev. 9, 151-160 (2000).
Boyd, N., Martin, L., Stone, J., Little, L., Minkin, S. & Yaffe, M. A longitudinal study of the effects of menopause on mammographic features. Cancer Epidemiol. Biomarkers Prev. 11, 1048-53 (2002).
Clifford, A. J., Ebeler, S. E., Ebeler, J. D., Bills, N. D., Hinrichs, S. H., Teissedre, P. L. & Waterhouse A. L. Delayed tumor onset in transgenic mice fed an amino acid-based diet supplemented with red wine solids. Am. J. Clin. Nutr. 64(5), 748-756 (1996).
Bhat, H. P., Lantvit, D., Christov, K., Mehta, R. G., Moon, R. C. & Pezzuto J. M. Estrogenic and antiestrogenic properties of resveratrol in mammary tumor models. Cancer Res. 61(20), 7456-7463 (2001).
Singletary, K. W. & Gapstur S. M. Alcohol and breast cancer: Review of epidemiologic and experimental evidence and potential mechanisms. JAMA, 286, 2143-2151 (2001).
Eng, E. T., Williams, D., Mandava, U., Kirma, N., Tekmal, R. R. & Chen, S. Anti-aromatase chemical in red wine. Ann. N.Y. Acad. Sci. 963, 239-246 (2002).
Eng, E. T. Ye, J., Williams, D., Phung, S., Moore, R., Young, M., Gruntmanis, U., Braunstein, G., & Chen, S. Suppression of Estrogen Biosynthesis by Procyanidin Dimers in Red Wine and Grape Seeds. Cancer Res., 63, 8516-8522 (2003).
Agarwal, C., Sharma, Y. & Agarwal, R. Anti-carcinogenic effect of a polyphenolic fraction isolated from grape seeds in human prostate carcinoma DU145 cells: modulation of mitogenic signaling and cell cycle regulators and induction of G1 arrest and apoptosis. Molecular Carcinogenesis 28, 129-138 (2000a).
Agarwal, C., Sharma, Y., Zhao, J. & Agarwal, R. A polyphenolic fraction from grape seeds causes irreversible growth inhibition of breast carcinoma MDA-MB468 cells by inhibiting mitogen-activated protein kinases activation and inducing G₁ arrest and differentiation. Clinical Cancer Res. 6, 2921-2930 (2000b).
Zhao, J., Wang, J., Chen, Y. & Agarwal, R. Anti-tumor-promoting activity of a polyphenolic fraction isolated from grape seeds in the mouse skin two-stage initiation-promotion protocol and identification of procyanidin B5-3′-gallate as the most effective antioxidant constituent. Carcinogenesis 20(9), 1737-1745 (1999).
Ye, X., Krohn, R. L., Liu, W., Joshi, S. S., Kuszynski, C. A., McGinn, T. R., Bagchi, M., Preuss, H. G., Stohs, S. J. & Bagchi, D. The cytotoxic effects of a novel IH636 grape seed proanthocyanidin extract on cultured human cancer cells. Mol. Cell Biochem. 196, 99-108 (1999).

1. A method of suppressing estrogen biosynthesis in a subject in need thereof, wherein the method comprises administering a therapeutically effective amount of a composition comprising procyanidin B.

2. The method of claim 1, wherein the procyanidin B is a procyanidin B dimer.

3. The method of claim 2, where in the procyanidin B dimer has a structure of 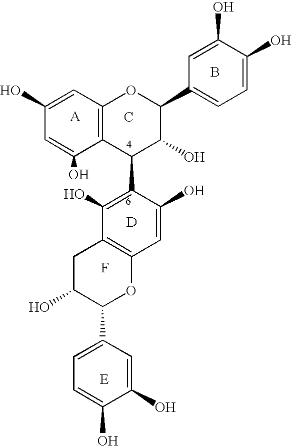
or stereoisomers thereof, or a prodrug thereof, or a pharmaceutically acceptable salt thereof.

4. The method of claim 1, wherein in subject in need thereof has a breast tumor, hormone-dependent cancer, or hormone imbalance.

5. The method of claim 4, wherein the overproduction of estrogen contributes to the condition of hormone-dependent cancer or hormone imbalance.

6. The method of claim 1, wherein the procyanidin B is purified.

7. The method of claim 1, wherein the procyanidin B is a component of a composition comprising a pharmaceutically acceptable carrier.

8. The method of claim 1, wherein the procyanidin B is a component of a composition comprising grape seed extract or red wine.

9. The method of claim 8, wherein the composition comprising grape seed extract or red wine is administered to a human.

10. The method of claim 9, wherein the grape seed extract is administered in an amount of between 50-500 mg/day.

11. The method of claim 9, wherein the grape seed extract is administered in an amount of between 200-300 mg/day.

12. A method of treating breast tumors, hormone-dependent cancer, or hormone imbalance, comprising administering a therapeutically effective amount of a composition comprising procyanidin B.

13. The method of claim 12, wherein the procyanidin B is a procyanidin B dimer.

14. The method of claim 12, where in the procyanidin B dimer has a structure of 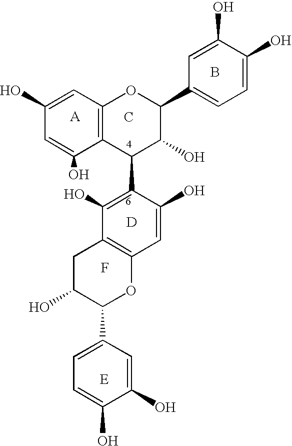
or stereoisomers thereof, or a prodrug thereof, or a pharmaceutically acceptable salt thereof.

15. The method of claim 12, wherein the overproduction of estrogen contributes to the condition of a breast tumor, hormone-dependent cancer or hormone imbalance.

16. The method of claim 12, wherein the procyanidin B is purified.

17. The method of claim 12, wherein the procyanidin B is a component of a composition comprising grape seed extract or red wine.

18. The method of claim 17, wherein the composition comprising grape seed extract or red wine is administered to a human and the amount of composition administered is between 50-500 mg/day.

19. The method of claim 17, wherein the composition comprising grape seed extract or red wine is administered to a human and the amount of composition administered is between 200-300 mg/day.

20. A method of suppressing estrogen biosynthesis in a human in need thereof, wherein the method comprises administering a therapeutically effective amount of a composition comprising procyanidin B dimer or stereoisomers thereof, or a prodrug thereof, or a pharmaceutically acceptable salt thereof in therapeutically effective carrier.
